(19) United States
(12) Patent Application Publication
(10) Pub. No.: US 2023/0329883 A1
(43) Pub. Date: Oct. 19, 2023

(54) Hydrotherapy Systems And Methods Of Use Of Said Systems

(71) Applicant: United States Government as Represented by the Department of Veterans Affairs, Washington, DC (US)

(72) Inventors: William Brian Layman, River Ridge, LA (US); Carly Harad, Irvine, CA (US); Clementine Furber, Jackson, WY (US); Gabrielle Sherer, Owings Mills, MD (US)

(21) Appl. No.: 18/300,786

(22) Filed: Apr. 14, 2023

Related U.S. Application Data
(60) Provisional application No. 63/331,471, filed on Apr. 15, 2022.

(51) Int. Cl. A61F 2/60; A61F 2/80

(52) U.S. Cl. CPC A61F 2/60; A61F 2/80; A61F 2002/607; A61F 2002/5075

(57) ABSTRACT

A system for providing hydrotherapy includes an attachment coupling structure that is configured to be secured to a bottom end portion of a socket that is configured to receive a residual limb of a subject. A fluid resistance attachment is coupled to the attachment coupling structure. The attachment coupling structure is configured to retain the fluid resistance attachment in a plurality of positions relative to the socket. The plurality of positions are rotationally offset relative to each other about an axis.

### CROSS-REFERENCE TO RELATED APPLICATION

This application claims priority to and the benefit of the filing date of U.S. Provisional Patent Application No. 63/331,471, filed Apr. 15, 2022, the entirety of which is hereby incorporated by reference herein.

### FIELD

This disclosure relates to systems and methods for enabling hydrotherapy for individuals with residual limbs.

### BACKGROUND

Physical therapy patients with residual limbs, such as below knee amputations, benefit from strengthening their residual limbs. Such strengthening can improve use of prosthetics and keep their residual limbs healthy. Many individuals with below knee amputations have other medical conditions such as diabetes or peripheral vascular disease that make it difficult and potentially dangerous to perform exercises on land. In hydrotherapy, water surrounds the individuals, making the body more buoyant, allowing for easier movement and reducing risk of injury. The individuals moves through the water, and the water provides resistance to movement. Conventionally, hydrotherapy is limited to the resistance of the water against the residual limb. Once the individuals develops enough strength to easily overcome this resistance, further muscular development becomes difficult.

Accordingly, a need exists for providing adjustable resistance to residual limbs. Still further, a need exists for adjusting a direction in which the resistance to movement is directed.

### SUMMARY

Disclosed herein, in one aspect, is a system for providing hydrotherapy. The system includes an attachment coupling structure that is configured to be secured to a bottom end portion of a socket, the socket being configured to receive a residual limb of a subject. A fluid resistance attachment is coupled to the attachment coupling structure. The attachment coupling structure is configured to retain the fluid resistance attachment in a plurality of positions relative to the socket. The plurality of positions are rotationally offset relative to each other about an axis.

In one aspect, a kit includes an attachment coupling structure that is configured to be secured to a bottom end portion of a socket, the socket being configured to receive a residual limb of a subject. A plurality of fluid resistance attachments are configured for selective removable coupling to the attachment coupling structure. Each fluid resistance attachment of the plurality of fluid resistance attachments is configured to provide a different amount of resistance upon displacement through water at an equal movement rate.

In one aspect, a method includes coupling a first fluid resistance attachment to an attachment coupling structure in a first rotational position relative to the attachment coupling structure. The attachment coupling structure is secured to a socket that is configured to receive a residual limb of a subject. The first fluid resistance attachment is moved from the first rotational position to a second rotational position that is offset from the first rotational position about an axis.

Additional advantages of the disclosed system and method will be set forth in part in the description which follows, and in part will be understood from the description, or may be learned by practice of the disclosed system and method. The advantages of the disclosed system and method will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed apparatus, system, and method and together with the description, serve to explain the principles of the disclosed apparatus, system, and method.

FIG. 1 is a perspective view of an exemplary attachment coupling structure for a system for providing hydrotherapy as disclosed herein.

FIG. 2 is a side view of the attachment coupling structure of FIG. 1, with internal features shown in broken lines.

FIG. 3 is a bottom view of the attachment coupling structure of FIG. 1.

FIG. 4 is a top view of the attachment coupling structure of FIG. 1.

FIG. 5 is a top view of a biasing element of the attachment coupling structure of FIG. 1.

FIG. 6 is a perspective view of the biasing element of FIG. 5.

FIG. 7 is a side view of the biasing element of FIG. 5.

FIG. 8 is a perspective view of a receptacle of the attachment coupling structure of FIG. 1.

FIG. 9A is a side view of the receptacle of FIG. 8. FIG. 9B is a cross sectional view of the receptacle of FIG. 9A, taken in the plane A-A.

FIG. 10 is a bottom view of the receptacle of FIG. 8.

FIG. 11 is a perspective view of an exemplary fluid resistance attachment comprising a fin, in accordance with embodiments disclosed herein.

FIG. 12 is a proximal end view of the exemplary fluid resistance attachment of FIG. 11.

FIG. 13 is a perspective view of an exemplary fluid resistance attachment comprising a fin, in accordance with embodiments disclosed herein.

FIG. 14 is a proximal end view of the exemplary fluid resistance attachment of FIG. 13.

FIG. 15 is a perspective view of an exemplary fluid resistance attachment comprising a fin, in accordance with embodiments disclosed herein.

FIG. 16 is a proximal end view of the exemplary fluid resistance attachment of FIG. 15.

FIG. 17 is a partial exploded view of an exemplary system for providing hydrotherapy as disclosed herein.

FIG. 18 is an assembled view of the system for providing hydrotherapy of FIG. 17.

FIG. 19 illustrates rotational alignment and coupling of an exemplary fluid resistance attachment to an exemplary attachment coupling as disclosed herein.

FIG. 20 shows another exemplary embodiment of an attachment coupling structure as disclosed herein.

FIG. 21 shows an exploded view of an exemplary attachment coupling structure with the biasing structure flipped 180 degrees to show the springs.

FIG. 22 shows elements of an exemplary kit as disclosed herein.

### DETAILED DESCRIPTION

The disclosed system and method may be understood more readily by reference to the following detailed description of particular embodiments and the examples included therein and to the Figures and their previous and following description.

It is to be understood that the terminology used herein is for the purpose of describing particular embodiments only and is not intended to limit the scope of the present invention which will be limited only by the appended claims.

It must be noted that as used herein and in the appended claims, the singular forms “a,” “an,” and “the” include plural references unless the context clearly dictates otherwise. Thus, for example, reference to “a radial projection” includes one or more of such radial projections, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Optionally, in some aspects, when values or characteristics are approximated by use of the antecedents “about,” “substantially,” or “generally,” it is contemplated that values within up to 15%, up to 10%, up to 5%, or up to 1% (above or below) of the particularly stated value or characteristic can be included within the scope of those aspects.

Unless defined otherwise, all technical and scientific terms used herein have the same meanings as commonly understood by one of skill in the art to which the disclosed apparatus, system, and method belong. Although any apparatus, systems, and methods and materials similar or equivalent to those described herein can be used in the practice or testing of the present apparatus, system, and method, the particularly useful methods, devices, systems, and materials are as described.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

As used herein, “proximal” refers to a position or direction near or towards the residual limb of an individual or patient.

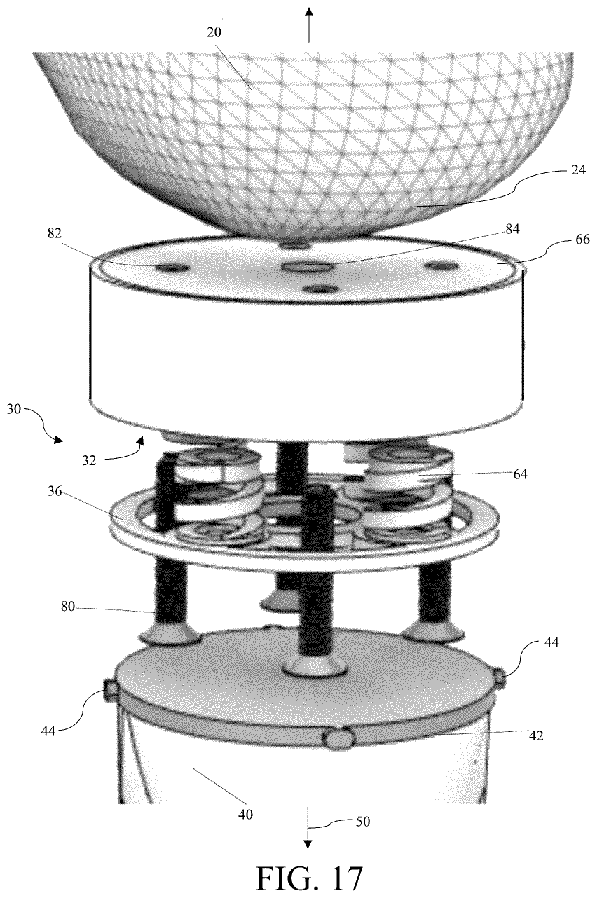

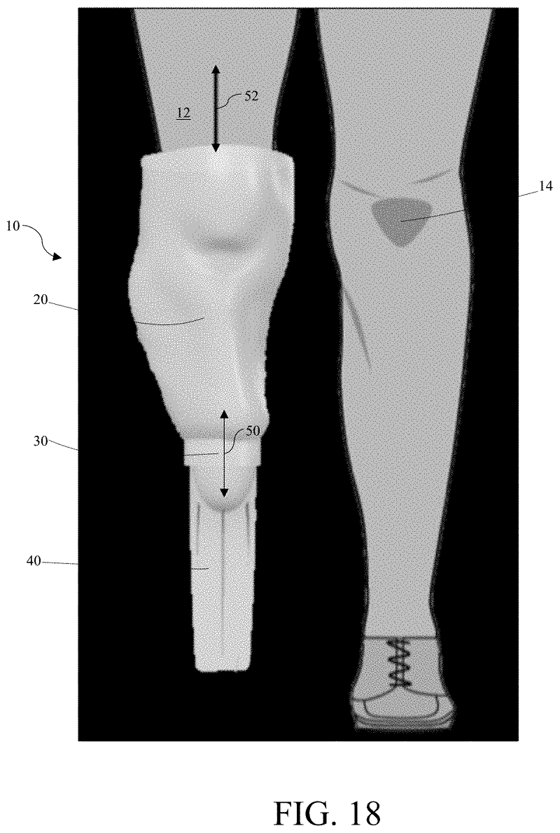

Disclosed herein, with reference to FIGS. 17 and 18, is a system 10 for providing hydrotherapy, which can include any activity performed in water to assist in rehabilitation and recovery. For example, the system 10 can be used by above- or below-the-knee amputees to strengthen a residual limb 12. In further aspects, the system 10 can be used for above or below the elbow residual limbs. Optionally, a user can have an intact limb 14 that is opposed to the residual limb 12 (for example, a left leg with a residual limb and a right leg with an intact limb). According to various aspects, the system 10 can enable positioning of the fluid resistance attachments in different rotational positions as well as coupling of different fluid resistance attachments. In this way, the system can be configured for different types of training and movements (e.g., during hydrotherapy activities) and can be adapted for providing different resistance levels.

The system 10 can comprise a socket 20 that is configured to receive the residual limb 12 (e.g., a residual limb below a knee). As used herein, a residual limb should be understood to include a portion of a limb remaining after amputation as well as a portion of a limb that is atypically or partially developed. Thus, the residual limb 12 can be, but need not be, a result of amputation.

The socket 20 can have a bottom end portion 24. An attachment coupling structure 30 can be secured to the bottom end portion 24 of the socket 20. A fluid resistance attachment 40 can be coupled to the attachment coupling structure 30. The fluid resistance attachment 40 can be configured to provide a resistance upon movement through water.

The attachment coupling structure 30 can be configured to retain the fluid resistance attachment in a plurality of positions relative to the socket 20. The plurality of position can be rotationally offset relative to each other about an axis 50. In various aspects, the plurality of positions can be even angular displacements such as, for example, every 18 degrees, or about 18 degrees, or every 90 degrees, or about every 90 degrees. In various aspects, the plurality of positions can comprise 2, 3, 4, 5, 6, 7, 8, 9, 10, 11, 12, 13, 13, 15, 16, 17, 18, 19, 20, or more different rotational positions. Optionally, the plurality of positions can consist of two positions that are 90 degrees, or about 90 degrees apart.

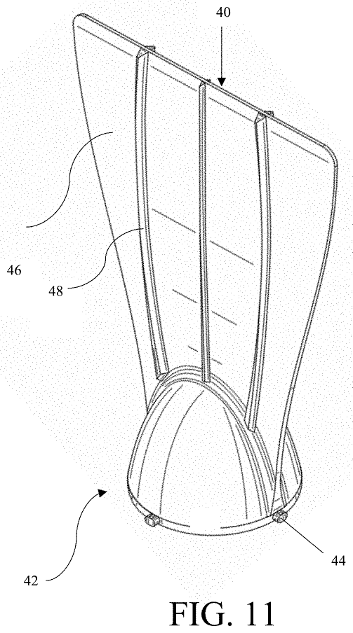

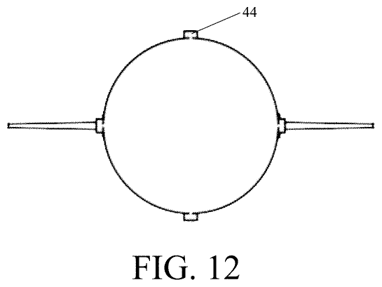

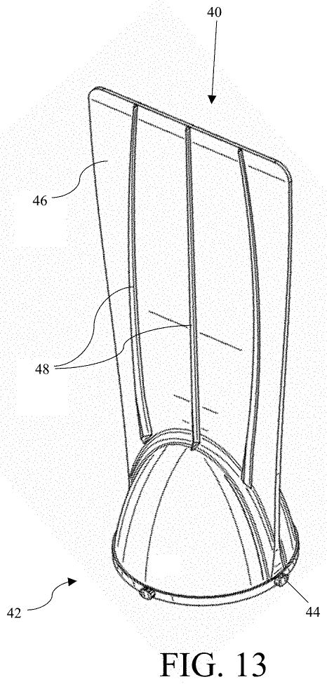

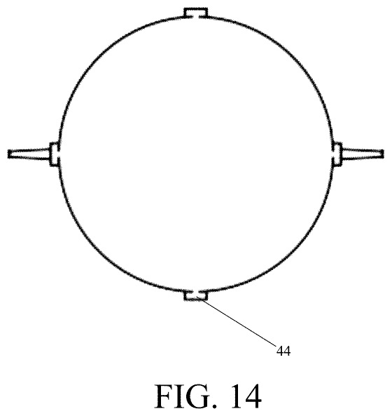

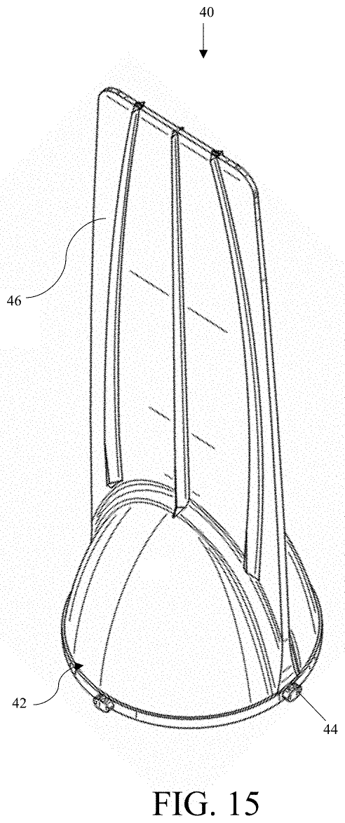

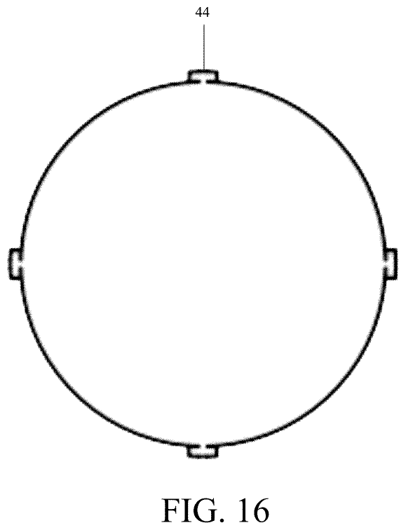

Referring to FIGS. 11-16, in exemplary aspects, the fluid resistance attachment 40 can comprise a fin 46. The fin 46 can optionally comprise stabilizing ribs 48. In various aspects, the fin 46 can have a flat (e.g., planar or generally planar) surface. In further aspects, the fin 46 can have a curved surface (e.g., curved about an axis 54 that is perpendicular to the axis 50). In further aspects, the fluid resistance attachment 40 can comprise a mesh, a scoop (e.g., a bowl-shape), a parachute, an airfoil, or tail (e.g., a fish-like tail), a plurality of ribbons, one or a plurality of stiff paddles, combinations thereof, and the like. Optionally, the fluid resistance attachment 40 can be weighted. In further aspects, the fluid resistance attachment 40 can comprise a buoyant (e.g., floatable) material. The fluid resistance attachment can be hollow or solid, and stiff or flexible. Optionally, the fluid resistance attachment 40 can comprise a material that changes flexibility depending on its temperature. In exemplary aspects, the fluid resistance attachment 40 can be hand-moldable, and/or movable about and between multiple configurations (e.g., flat and curved). For example, the fluid resistance attachment 40 can be movable about and between a first configuration, in which the fluid resistance attachment is flat, and as second configuration in which the fluid resistance attachment is curved. In this way, the fluid resistance attachment can be adjustable to provide different amounts of resistance. In still further aspects, the fluid resistance attachment can comprise added or removable accessories to change its properties (e.g., its surface area or drag in water). For example, in some aspects, one or more ribbons, projections, or a mesh can be attached to the rest of the fluid resistance attachment to change the resistance of the fluid resistance attachment as it moves through water. The attachment can be secured to the rest of the fluid resistance attachment via fasteners (e.g., screws, bolts, hooks, loops straps, etc.). Generally, the fluid resistance attachment 40 can comprise one or more surfaces that engage with fluid (e.g., water) to increase an amount of fluid resistance acting on the individuals.

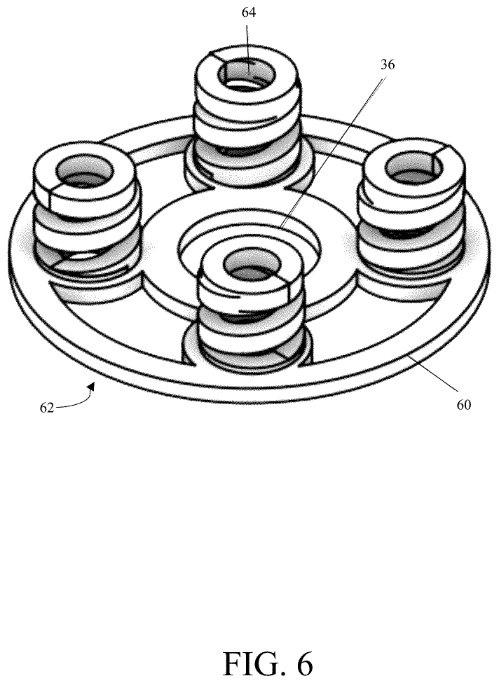

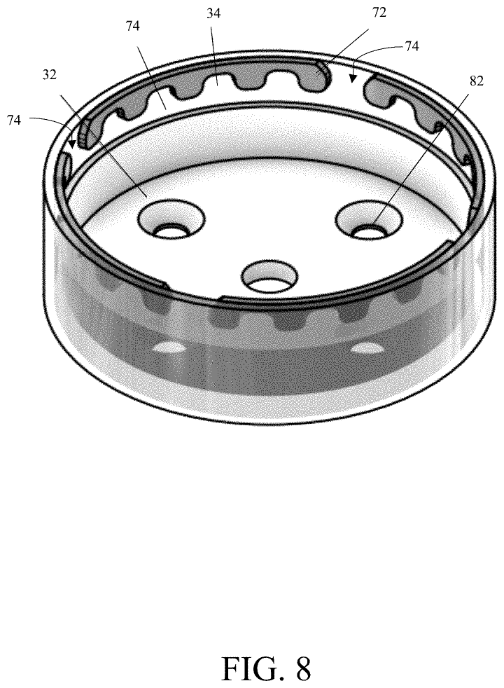

In some aspects, the fluid resistance attachment 40 can comprise a proximal end portion 42. The proximal end portion 42 can have at least one radial projection 44. Referring also to FIGS. 6-9B, the attachment coupling structure 30 can comprise a receptacle 32 that is configured to receive the proximal end portion 42 of the fluid resistance attachment 40. A plurality of catches 34 can be positioned within the receptacle and can be spaced circumferentially about the axis 50. Each catch 34 of the plurality of catches can be configured to receive a respective radial projection 44 of the at least one radial projection of the proximal end portion 42 of the fluid resistance attachment 40. The fluid resistance attachment 40 can be inhibited from rotation about the axis 50 when each respective radial projection 44 of the at least one radial projection is received by a respective catch 34 of the plurality of catches. A biasing element 36 can be configured to axially bias the fluid resistance attachment 40 in a direction away from the socket 20. In this way, the biasing element 36 can bias the fluid resistance attachment 40 so that the radial projection(s) 44 are received within the catches 34. In some optional aspects, the attachment coupling structure 30 can comprise polymer (e.g., nylon), carbon, a composite material, or metal, or combinations thereof. The attachment coupling structure 30 can be durable and waterproof (e.g., resistant to oxidation or degradation in water). The attachment coupling structure 30 can comprise smooth surfaces to inhibit damage to the fluid resistance attachment 40 and permit smooth movement between the fluid resistance attachment and the attachment coupling structure.

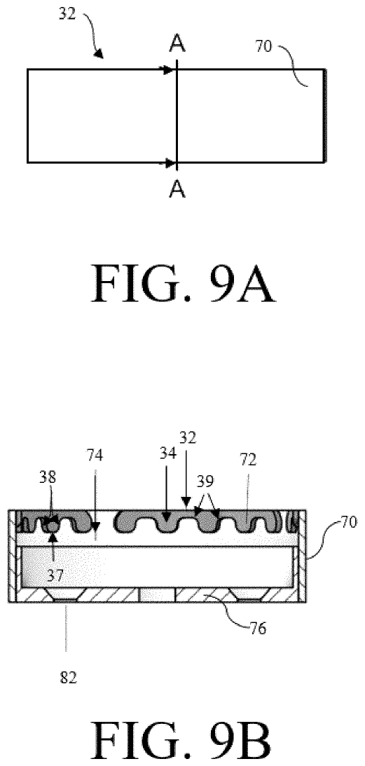

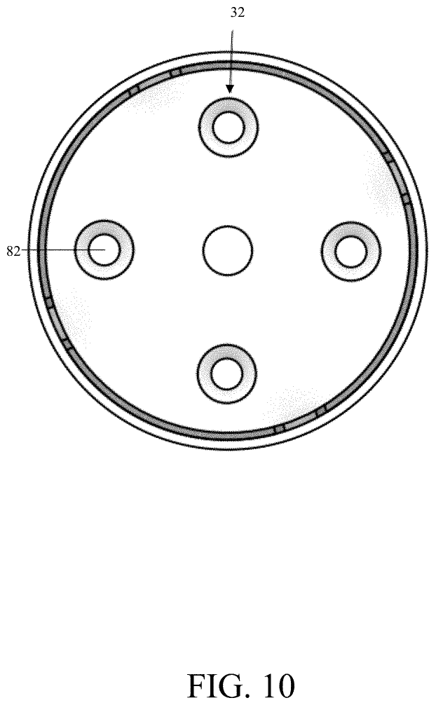

Referring to FIG. 9B, in exemplary aspects, each catch 34 of the plurality of catches can comprise a recess 37 defined between opposed sidewalls 38. For example, the attachment coupling 30 can comprise an outer wall 70 that at least partially defines the receptacle 32. One or a plurality of radially inwardly extending projections 72 can extend inwardly from the outer wall 70 into the recess. The radially inwardly extending projections 72 can define the catches 34 (e.g., recesses 37). For example, each radially inwardly extending projection 72 can define one, two, three, four (as shown), five, or more catches 34.

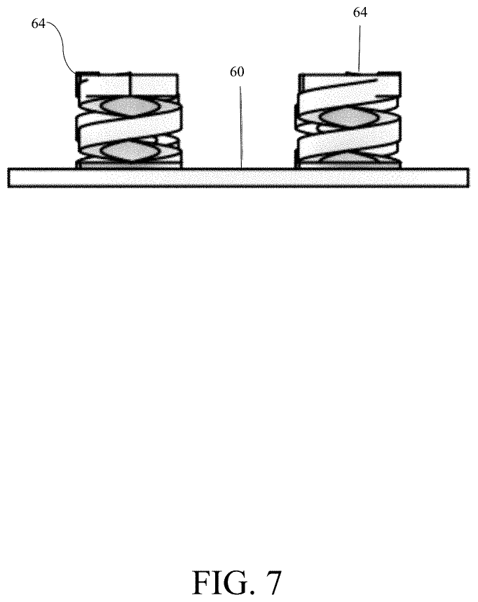

Referring to FIGS. 6-7, in some aspects, the biasing element 36 can comprise a body 60 (e.g., a disk or concentric annuluses) defining a biasing surface 62 that biases against the fluid resistance attachment 40 and a plurality of springs 64 (e.g., four springs) that are coupled to the body 60. In some aspects, the body 60 can define openings to reduce its weight and material usage, as can be advantageous for 3D printing. The plurality of springs 64 can be circumferentially spaced about the axis 50. For example, the circumferentially spaced springs 64 can evenly distribute a biasing force across the fluid resistance attachment 40 to permit smooth movement of the fluid resistance attachment. In further aspects, the biasing element 36 can comprise a single spring 64. Optionally, each spring 64 can be a coil spring. In still further optional aspects, the body 60 can be omitted. In some optional aspects, the springs 64 (optionally, the entire biasing element) can comprise polymer (e.g., Nylon PA 12). In some optional aspects, the springs 64 (optionally, the entire biasing element 36) can be formed via additive manufacturing (e.g., 3D printing). In various optional aspects, the springs 64 can have rectangular cross sections, triangular cross sections, or circular cross sections.

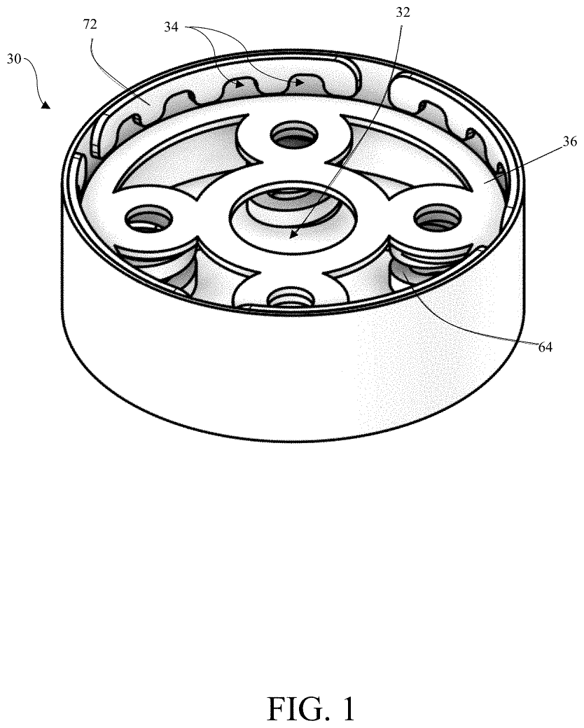

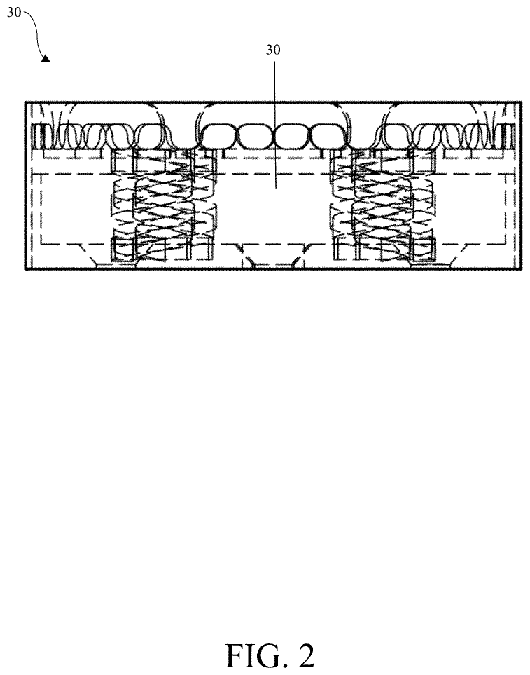

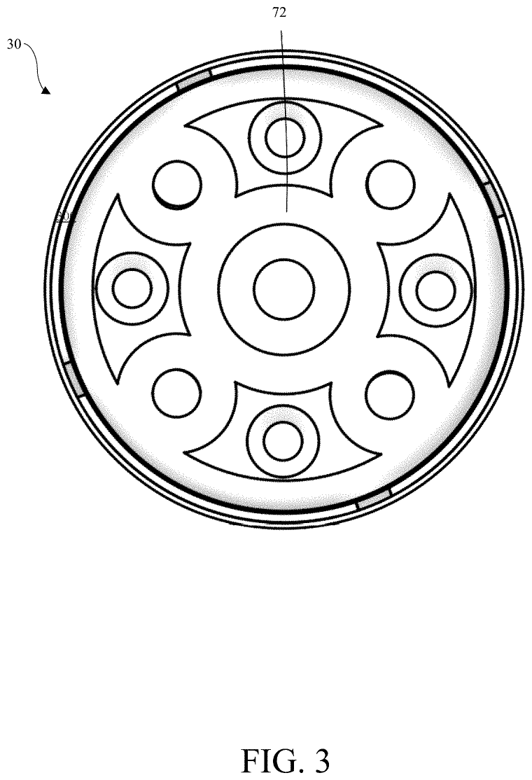

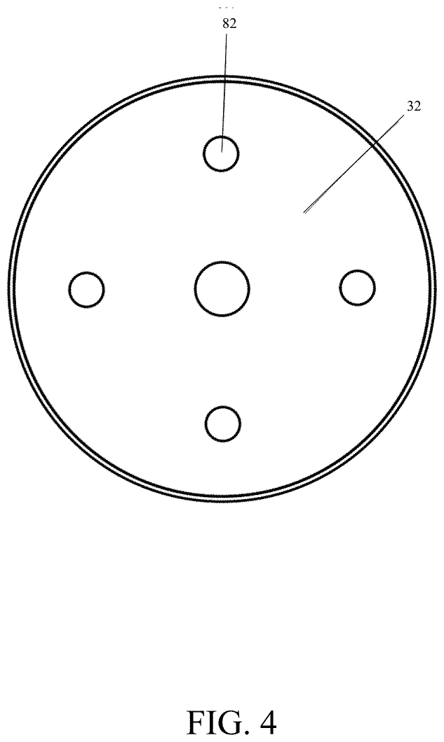

Referring to FIGS. 1 and 9B, in exemplary aspects, a bottom cap 76 can couple to the outer wall 70 so that the bottom cap and outer wall cooperate to define the receptacle 32. For example, the bottom cap 76 can couple to the outer wall 70 via press-fit, adhesive, or integral formation. In some optional aspects, the biasing element 36 can be axially retained between the radially inwardly extending projections 72 and the bottom cap 76.

For each radial projection 44, the attachment coupling structure 30 can comprise a respective slot 74 that extends between sequential catches 34 of the plurality of catches along the axis 50. In this way, the slots 74 can permit the radial projections 44 to pass by the plurality of catches 34 as the proximal end portion is received into the receptacle 32. Accordingly, the slots 74 can have the same circumferential spacing as the radial projections 44 so that the slots 74 can simultaneously receive the radial projections. In exemplary aspects, the slots 74 can be defined between adjacent radially inwardly extending projections 72. Optionally, the slots can have flared openings in the direction away from the socket to guide the radial projections 44 into the slots.

In various exemplary aspects, the attachment coupling structure 30 can comprise any suitable structure for securing the fluid resistance attachment 40 to the socket 20. For example, in some aspects, the attachment coupling structure 30 can comprise a plurality of openings (e.g., threaded openings) that receive respective screws. The screws can extend through the fluid resistance attachment 40 and secure the fluid resistance attachment to the attachment coupling structure 30. In some aspects, the attachment coupling structure 30 can comprise an opening that is configured to receive the proximal end portion 42 of the fluid resistance attachment 40, and the opening can be adjusted to tighten down on the proximal end portion 42 of the fluid resistance attachment 40. For example, the attachment coupling structure can further comprise a lever (e.g., a quick-release lever with an eccentric pivotal axis) that can be actuated to reduce an operative circumference of the opening to tighten down on the proximal end portion 42 of the fluid resistance attachment 40. In still further aspects, the attachment coupling structure 30 can comprise one or more straps, locking pins, hooks, clamps, threaded fasteners, or catches for securing the fluid resistance attachment 40.

The socket 20 can be configured to receive the residual limb 12 along a second axis 52. In some optional aspects, the second axis 52 can be parallel to, or generally parallel to, the axis 50. For example, in some aspects, the second axis 52 can be within 15 degrees, or within 10 degrees, or within 5 degrees, or within 1 degree of the axis 50.

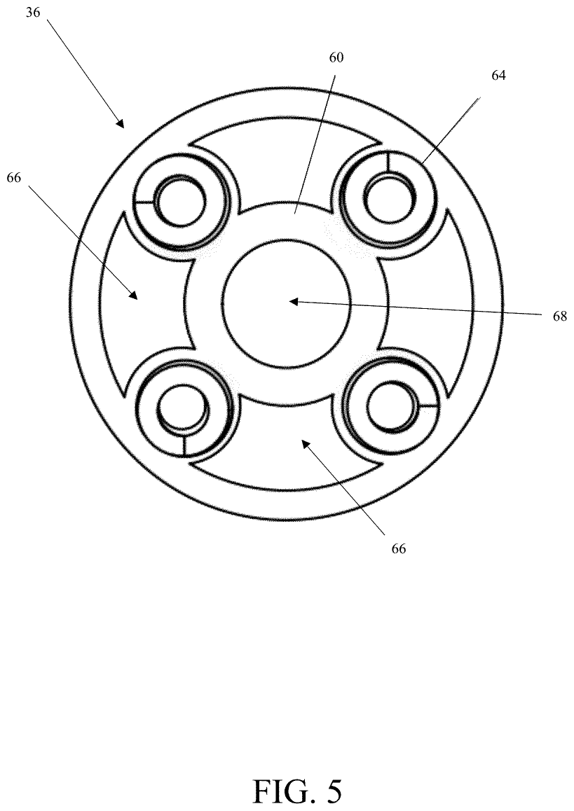

In some optional aspects, the attachment coupling structure 30 can be secured to the socket 20 by integral formation. In alternative aspects, and with reference to FIG. 17, the attachment coupling structure 30 can be secured to the socket 20 by at least one fastener 80 (optionally, a plurality of fasteners). For example, a plurality of screws (e.g., four screws, as shown) can extend through holes 82 in the coupling structure 30 and into the socket 20. In exemplary aspects, the screws can be M4 screws, as are commonly used in prostheses. Referring also to FIG. 5, the body 60 of the biasing element 36 can define openings 66 (e.g. between concentric annuluses) that permit the fasteners 80 and a tool (e.g., screw driver) to extend therethrough for coupling the attachment coupling structure 30 to the socket. In various further aspects, the at least one fastener 80 can comprise hooks, snaps, pins, adhesive, combinations thereof, or the like. The attachment coupling structure 30 can be configured to couple to any socket construction, or any suspension construction, as is known in the art. For example, an exemplary socket can have a hole pattern that is configured to receive screws, and the holes 82 can be configured to match said hole pattern. In yet further aspects, the socket can comprise a pin lock socket, as is known in the art, in which a pin extends from the socket. The attachment coupling structure 30 can define openings to receive the pin. For example, the bottom cap 76 can define an opening 84, and the body 60 of the biasing element 36 can define a center opening 68 that are each configured to receive therethrough the pin of the socket. Accordingly, in some aspects, the attachment coupling structure 30 can be configured to couple to both a first socket having a hole pattern for receiving a plurality of screws and a second socket that comprises a pin lock. In still further aspects, the attachment coupling structure 30 can couple to the socket 20 via one or more straps, locking pins, hooks, clamps, threaded fasteners, or catches.

### Method of Use

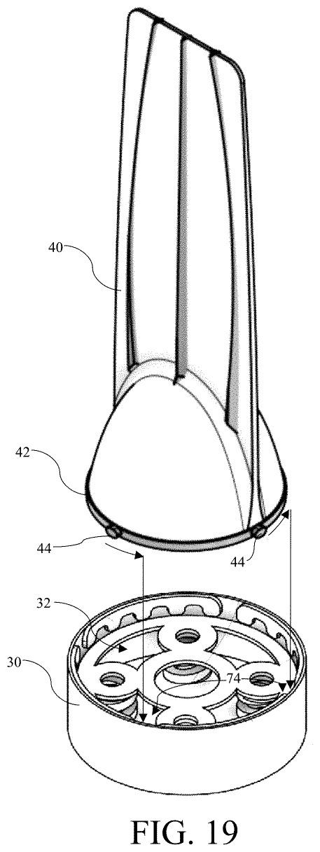

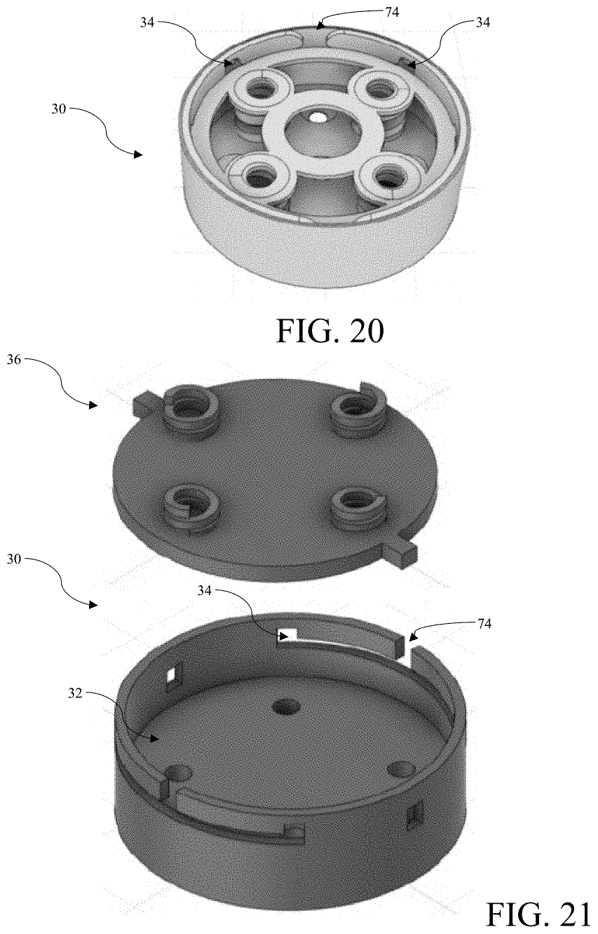

A first fluid resistance attachment 40 can be coupled to the attachment coupling structure 30 in a first rotational position relative to the attachment coupling structure. For example, as illustrated in FIG. 19, each radial projection 44 of the at least one radial projection can be aligned with a respective slot 74, and the proximal end portion 42 of the first fluid resistance attachment 40 can be inserted into receptacle 32. The proximal end portion 42 of the first fluid resistance attachment 40 can be inserted until the radial projection(s) 44 are moved past the catches 34 along the axis 50. The first fluid resistance attachment 40 can then be rotated about the axis 50 so that the first fluid resistance attachment is oriented in a desired position. The first fluid resistance attachment 40 can then be released, and the biasing element 36 can bias the first fluid resistance attachment until the radial projections 44 are received within respective catches 34. In some aspects, guide surfaces 39 can be positioned between the catches 34, the guide surfaces being rounded or sloping toward the adjacent catches to guide the radial projections 44 into the catches so that the radial projections do not get stuck between catches. In this way, as the biasing element 36 biases the radial projections 44 toward the catches, the radial projections 44 can align with, and be received within, catches 34.

It is contemplated that the first fluid resistance attachment 40 can be positioned in a desired orientation. For example, the first fluid resistance attachment 40 can be oriented to provide a maximum resistance against a direction of motion. Thus, for example, first fluid resistance attachment 40 can comprise a fin 46. The fin 46 can have an operational surface that is oriented perpendicular to a direction of motion. For example, for front-to-rear kicking, the operational surface of the fin 46 can be oriented perpendicular to a front-to-rear (sagittal) axis of the user (into and out of the page in FIG. 18). For side-to-side kicking (movement left and right in FIG. 18), the operational surface of the fin 46 can be oriented parallel to the front-to-rear (sagittal) axis of the user.

In exemplary aspects, with the first fluid resistance attachment 40 in the first rotational position, the system 10 can be used in hydrotherapy. For example, the system 10 can be used for hip abduction, hip flexion, hip adduction, hip extension, hamstring curls, or high hip rotation.

The first fluid resistance attachment 40 can be moved from the first rotational position to a second rotational position that is offset from the first rotational position about an axis. For example, the first fluid resistance attachment 40 can be pushed proximally (inwardly into the receptacle 32, against the biasing element 36) until the radial projection(s) 44 are moved past the catches 34 along the axis 50. The first fluid resistance attachment 40 can be rotated to the second position about the axis 50 and then released so that the biasing element 36 biases the radial projection(s) 44 into respective catches 34.

With the first fluid resistance attachment 40 in the second rotational position, the system 10 can be used in hydrotherapy.

In further aspects, the first fluid resistance attachment 40 can be decoupled from the attachment coupling structure. For example, the first fluid resistance attachment 40 can be pushed proximally (inwardly into the receptacle 32, against the biasing element 36) until the radial projection(s) 44 are moved past the catches 34 along the axis 50. The first fluid resistance attachment 40 can be rotated to align the radial projection(s) 44 with respective slot(s). The first fluid resistance attachment 40 can then be withdrawn from the receptacle 32 along the axis 50.

A second fluid resistance attachment 40 can then be coupled to the attachment coupling structure in the manner described for the first fluid resistance attachment. The second fluid resistance attachment can be different from the first fluid resistance attachment. For example, the second fluid resistance attachment can comprise a fin having a larger or smaller surface area than a fin of the first fluid resistance attachment.

### Kit

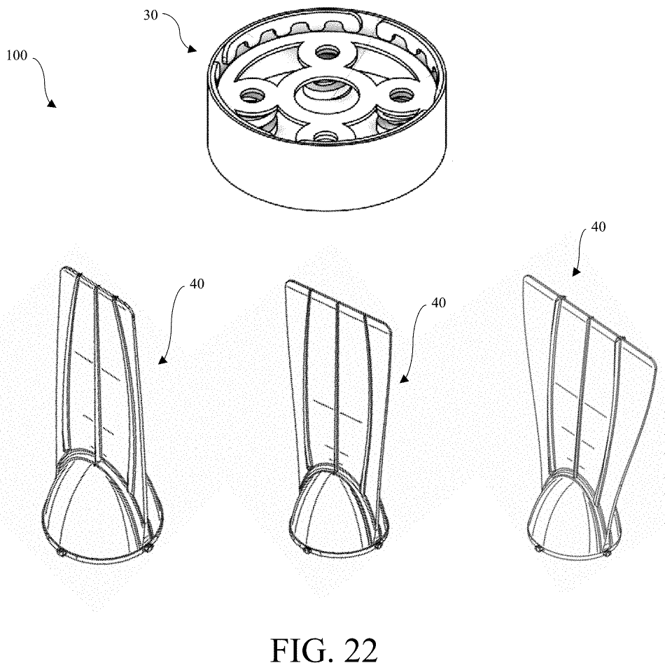

Referring to FIG. 22, a kit 100 can comprise an attachment coupling structure 30 that is configured to be secured to the bottom end portion 24 of the socket 20. The kit 100 can further comprise a plurality of fluid resistance attachments 40 configured for selective removable coupling to the attachment coupling structure. Each fluid resistance attachment 40 of the plurality of fluid resistance attachments can be configured to provide a different amount of resistance upon displacement through water at an equal movement rate. For example, the plurality of fluid resistance attachments can comprise a plurality of fins having different surface areas.

In some optional aspects, the kit can further comprise the socket 20.

In some optional aspects, the attachment coupling structure 30 can be configured to retain the fluid resistance attachment in a plurality of positions relative to the socket. The plurality of positions can be rotationally offset relative to each other about an axis 50. In further aspects, it is contemplated that the attachment coupling structure 30 can be configured to retain the fluid resistance attachment in only a single position relative to the socket.

### Exemplary Aspects

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A system for providing hydrotherapy, the system comprising:
an attachment coupling structure that is configured to be secured to a bottom end portion of a socket, wherein the socket is configured to receive a residual limb of a subject; and
a fluid resistance attachment that is coupled to the attachment coupling structure,
wherein the attachment coupling structure is configured to retain the fluid resistance attachment in a plurality of positions relative to the socket, wherein the plurality of positions are rotationally offset relative to each other about an axis.

Aspect 2: The system of aspect 1, wherein the fluid resistance attachment comprises a fin.

Aspect 3: The system of aspect 1 or aspect 2, wherein the fluid resistance attachment comprises a mesh.

Aspect 4: The system of any one of the preceding aspects, wherein the fluid resistance attachment comprises a proximal end portion, the proximal end portion having at least one radial projection, wherein the attachment coupling structure comprises:
a receptacle that is configured to receive the proximal end portion of the fluid resistance attachment;
a plurality of catches positioned within the receptacle and spaced circumferentially about the axis, wherein each catch of the plurality of catches is configured to receive a respective radial projection of the at least one radial projection of the proximal end portion of the fluid resistance attachment, wherein the fluid resistance attachment is inhibited from rotation about the axis when each respective radial projection of the at least one radial projection is received by a respective catch of the plurality of catches; and
a biasing element that is configured to axially bias the fluid resistance attachment in a first direction.

Aspect 5: The system of aspect 4, wherein each catch of the plurality of catches is defined by a respective recess defined between opposed sidewalls of the receptacle.

Aspect 6: The system of aspect 4 or aspect 5, wherein the biasing element comprises:
a body defining a biasing surface that biases against the fluid resistance attachment; and
a plurality of springs that are coupled to the body, wherein the plurality of springs are circumferentially spaced about the axis.

Aspect 7: The system of any one of aspects 4-6, wherein the attachment coupling structure comprises, for each radial projection of the proximal end portion of the fluid resistance attachment, a respective slot between sequential catches of the plurality of catches, wherein the respective slot is configured to receive a respective radial projection of the at least one radial projection along the axis.

Aspect 8: The system of aspect 7, wherein the attachment coupling comprises:
an outer wall that at least partially defines the receptacle; and
at least one radially inwardly extending projection that extends inwardly from the outer wall into the recess, wherein radially inwardly extending projections define the catches.

Aspect 9: The system of aspect 8, wherein the at least one radially inwardly extending projection comprises a plurality of radially inwardly extending projections, wherein adjacent radially inwardly extending projections of the plurality of radially inwardly extending projections define therebetween the respective slots.

Aspect 10: The system of any one of the preceding aspects, further comprising the socket, wherein the attachment coupling structure is secured to the bottom end portion of the socket, wherein the axis is a first axis, wherein the socket is configured to receive the residual limb along a second axis that is parallel to, or generally parallel to, the first axis.

Aspect 11: The system of any one of the preceding aspects, further comprising the socket, wherein the attachment coupling structure is secured to the socket by integral formation.

Aspect 12: The system of any one of the preceding aspects, further comprising the socket, wherein the attachment coupling structure is secured to the socket by at least one fastener.

Aspect 13: The system of any one of the preceding aspects, wherein the plurality of positions comprise at least 4 different positions.

Aspect 14: The system of any one of the preceding aspects, wherein the plurality of positions comprise about 16 different positions.

Aspect 15: The system of any one of the preceding aspects, wherein at least two adjacent positions of the plurality of positions are rotationally offset about the axis by about 18 degrees.

Aspect 16: The system of any one of the preceding aspects, wherein the attachment coupling structure is configured to couple to both:
a first socket having a hole pattern for receiving a plurality of screws; and
a second socket that comprises a pin lock.

Aspect 17: A kit comprising:
an attachment coupling structure that is integral to, or configured to be secured to, a bottom end portion of a socket that is configured to receive a residual limb of a subject; and
a plurality of fluid resistance attachments configured for selective removable coupling to the attachment coupling structure, wherein each fluid resistance attachment of the plurality of fluid resistance attachments is configured to provide a different amount of resistance upon displacement through water at an equal movement rate.

Aspect 18: The kit of aspect 17, wherein the attachment coupling structure is configured to retain the fluid resistance attachment in a plurality of positions relative to the socket, wherein the plurality of positions are rotationally offset relative to each other about an axis.

Aspect 19: The kit of aspect 17 or aspect 18, wherein the plurality of fluid resistance attachments comprise a plurality of fins having different surface areas.

Aspect 20: The kit of any one of aspects 17-19, further comprising the socket.

Aspect 21: A method comprising:
coupling a first fluid resistance attachment to an attachment coupling structure in a first rotational position relative to the attachment coupling structure, wherein the attachment coupling structure is secured to a socket that is configured to receive a residual limb of a subject;
moving the first fluid resistance attachment from the first rotational position to a second rotational position that is offset from the first rotational position about an axis.

Aspect 22: The method of aspect 21, further comprising:
and decoupling the first fluid resistance attachment from the attachment coupling structure;
coupling a second fluid resistance attachment to the attachment coupling structure.

Those skilled in the art will recognize, or be able to ascertain using no more than routine experimentation, many equivalents to the specific embodiments of the method and compositions described herein. Such equivalents are intended to be encompassed by the following claims.

What is claimed is:

1. A system for providing hydrotherapy, the system comprising:
an attachment coupling structure that is configured to be secured to a bottom end portion of a socket, wherein the socket is configured to receive a residual limb of a subject; and
a fluid resistance attachment that is coupled to the attachment coupling structure,
wherein the attachment coupling structure is configured to retain the fluid resistance attachment in a plurality of positions relative to the socket, wherein the plurality of positions are rotationally offset relative to each other about an axis.

2. The system of claim 1, wherein the fluid resistance attachment comprises a fin.

3. The system of claim 1, wherein the fluid resistance attachment comprises a mesh.

4. The system of claim 1, wherein the fluid resistance attachment comprises a proximal end portion, the proximal end portion having at least one radial projection, wherein the attachment coupling structure comprises:
a receptacle that is configured to receive the proximal end portion of the fluid resistance attachment;
a plurality of catches positioned within the receptacle and spaced circumferentially about the axis, wherein each catch of the plurality of catches is configured to receive a respective radial projection of the at least one radial projection of the proximal end portion of the fluid resistance attachment, wherein the fluid resistance attachment is inhibited from rotation about the axis when each respective radial projection of the at least one radial projection is received by a respective catch of the plurality of catches; and
a biasing element that is configured to axially bias the fluid resistance attachment in a first direction.

5. The system of claim 4, wherein each catch of the plurality of catches is defined by a respective recess defined between opposed sidewalls of the receptacle.

6. The system of claim 4, wherein the biasing element comprises:
a body defining a biasing surface that biases against the fluid resistance attachment; and
a plurality of springs that are coupled to the body, wherein the plurality of springs are circumferentially spaced about the axis.

7. The system of claim 4, wherein the attachment coupling structure comprises, for each radial projection of the proximal end portion of the fluid resistance attachment, a respective slot between sequential catches of the plurality of catches, wherein the respective slot is configured to receive a respective radial projection of the at least one radial projection along the axis.

8. The system of claim 7, wherein the attachment coupling comprises:
an outer wall that at least partially defines the receptacle; and
at least one radially inwardly extending projection that extends inwardly from the outer wall into the recess, wherein radially inwardly extending projections define the catches.

9. The system of claim 8, wherein the at least one radially inwardly extending projection comprises a plurality of radially inwardly extending projections, wherein adjacent radially inwardly extending projections of the plurality of radially inwardly extending projections define therebetween the respective slots.

10. The system of claim 1, further comprising the socket, wherein the attachment coupling structure is secured to the bottom end portion of the socket, wherein the axis is a first axis, wherein the socket is configured to receive the residual limb along a second axis that is parallel to, or generally parallel to, the first axis.

11. The system of claim 1, further comprising the socket, wherein the attachment coupling structure is secured to the socket by integral formation.

12. The system of claim 1, further comprising the socket, wherein the attachment coupling structure is secured to the socket by at least one fastener.

13. The system of claim 1, wherein the plurality of positions comprise at least 4 different positions.

14. The system of claim 1, wherein the plurality of positions comprise about 16 different positions.

15. The system of claim 1, wherein the attachment coupling structure is configured to couple to both:
a first socket having a hole pattern for receiving a plurality of screws; and
a second socket that comprises a pin lock.

16. A kit comprising:
an attachment coupling structure that is integral to, or configured to be secured to, a bottom end portion of a socket that is configured to receive a residual limb of a subject; and
a plurality of fluid resistance attachments configured for selective removable coupling to the attachment coupling structure, wherein each fluid resistance attachment of the plurality of fluid resistance attachments is configured to provide a different amount of resistance upon displacement through water at an equal movement rate.

17. The kit of claim 16, wherein the plurality of fluid resistance attachments comprise a plurality of fins having different surface areas.

18. The kit of claim 16, further comprising the socket.

19. A method comprising:
coupling a first fluid resistance attachment to an attachment coupling structure in a first rotational position relative to the attachment coupling structure, wherein the attachment coupling structure is secured to a socket that is configured to receive a residual limb of a subject;
moving the first fluid resistance attachment from the first rotational position to a second rotational position that is offset from the first rotational position about an axis.

20. The method of claim 19, further comprising:
decoupling the first fluid resistance attachment from the attachment coupling structure; and
coupling a second fluid resistance attachment to the attachment coupling structure.
